(19) United States
(12) Patent Application Publication
(10) Pub. No.: US 2026/0179373 A1
(43) Pub. Date: Jun. 25, 2026

(54) Data Classification Method and System Based on Deep Multipath Attention Adaptive Graph Convolutional Networks

(71) Applicant: Wenzhou University, Wenzhou City (CN)

(72) Inventors: Zhongyi HU, Wenzhou City (CN); Shuzhan ZHANG, Wenzhou City (CN); Lei XIAO, Wenzhou City (CN); Hui HUANG, Wenzhou City (CN); Wenhao WU, Wenzhou City (CN)

(73) Assignee: Wenzhou University, Wenzhou City (CN)

(21) Appl. No.: 19/432,060

(22) Filed: Dec. 23, 2025

(30) Foreign Application Priority Data
Dec. 24, 2024 (CN) 202411914971.7

(51) Int. Cl. G06V 10/82; G06N 3/042; G06N 3/0455; G06N 3/0464; G06V 10/764; G06V 10/77; G06V 10/774; G06V 10/776; G16H 50/70

(52) U.S. Cl. CPC G06V 10/82; G06N 3/042; G06N 3/0455; G06N 3/0464; G06V 10/764; G06V 10/7715; G06V 10/774; G06V 10/776; G16H 50/70; G06V 2201/031

(57) ABSTRACT

The present invention discloses a data classification method and system based on a deep multi-path attention adaptive graph convolutional network, belonging to the field of medical image processing technology. It involves acquiring and preprocessing rs-fMRI data to obtain BOLD sequences, constructing functional connectivity feature vectors as input to the DMAGCN model, and finally obtaining the optimal model for classification through five-fold cross-validation. The present invention ensures data quality through data preprocessing, laying the foundation for accurate analysis. The functional connectivity feature vectors effectively represent the data, and after inputting them into the model, the Transformer backbone network and MLP branch network can extract multi-source domain features. Combining this with the graph network utilizing non-imaging data, the model can learn rich features, enhancing its generalization and adaptability.

### INCORPORATION BY REFERENCE

This application claims priority to China Patent Application No. 202411914971.7 filed Dec. 24, 2025, the contents of which are hereby incorporated by reference in their entirety.

### TECHNICAL FIELD

The present invention relates to the technical field of medical image processing, and more specifically to a data classification method and system based on deep multipath attention adaptive graph convolutional networks.

### BACKGROUND TECHNOLOGY

Currently, Autism Spectrum Disorder (ASD) is a childhood neurodevelopmental disorder clinically characterized by stereotyped behaviors, narrow interests, and communication impairments. Early intervention in ASD leads to better outcomes; however, some ASD patients go undetected in childhood, with symptoms only observed in adolescence. Therefore, early prevention of ASD is essential.

In the past few decades, and especially in the last decade, advances in neuroimaging technology have provided a crucial step, enabling the measurement of functional and structural changes associated with ASD. Several methods based on resting-state functional magnetic resonance imaging (rs-fMRI) have been proposed for diagnosing various brain diseases, gaining widespread attention in the discovery and classification of ASD biomarkers. It is a key tool that can reveal brain dysfunction based on the blood oxygen level-dependent (BOLD) signal of a subject at rest. The Pearson correlation coefficient, calculated as functional connectivity by averaging the BOLD signal of two brain regions in rs-fMRI, has been widely used in computer-aided ASD diagnosis due to its effectiveness in identifying brain functional tissues and biomarkers in neuropsychiatric disorders. Most rs-fMRI-based ASD classification methods are developed using functional connectivity as a feature.

Deep learning has been successfully applied to disease diagnosis with remarkable results. However, human neural activity is characterized by uncertainty. This is because the process of acquiring rs-fMRI data clinically is subject to interference from noise inherent in the patient and equipment limitations, making it difficult to accurately reflect true neural activity. Secondly, ASD exhibits high heterogeneity, with significant individual differences in neural activity among different ASD patients. These factors introduce significant uncertainty into rs-fMRI data, hindering the construction of robust ASD-assisted diagnostic models. Simultaneously, clinical neural image datasets often suffer from small dataset sizes due to their expensive acquisition and time-consuming labeling. Therefore, in some studies, such as ASD diagnosis, multi-site rs-fMRI data are frequently merged to expand the dataset, leading to a second problem: in most cases, samples from different scanners or acquisition protocols do not follow the same distribution. Sun et al. minimized domain shift by aligning the second-order statistics of source and target domain distributions; Liu et al. mitigated marginal distribution differences between domains by adjusting the global structure of predicted multi-site data; Wang et al. aimed to reduce data distribution differences by determining a universal low-rank representation of data from multiple sites; Eslami et al. used autoencoders and single-layer perceptrons (SLPs) to improve the quality of extracted features and optimize model parameters. These methods are effective in addressing the problem of inconsistent dataset feature distributions, but their feature processing is not granular enough, potentially leading to misdiagnosis, which is a serious issue in the medical field.

Therefore, how to handle the classification of uncertain data is a problem urgently needing to be solved by those skilled in the art.

### SUMMARY OF THE INVENTION

In view of the above, the present invention provides a data classification method and system based on a deep multipath attention adaptive graph convolutional network (DMAGCN). A novel deep neural network—DMAGCN—is proposed, capable of handling uncertain data classification, comprising a backbone network and multiple branch networks. The backbone network is constructed using a Transformer to extract common features from different source domains. The branch networks are constructed using MLP and GCN, with a number equal to a number of source domains. These branch networks extract unique features from the source domains. The GCN, by constructing a population map, combines image and non-image data to extract multimodal information for model training. In the feature classification stage, the features obtained from each branch are concatenated, and the softmax function is used to obtain the final classification result, thereby achieving classification of uncertain data.

To achieve the above objectives, the present invention employs the following technical solution:

A data classification method based on a deep multipath attention adaptive graph convolutional network, comprising:
acquiring resting-state functional magnetic resonance imaging (fMRI) data from an autism brain imaging data exchange database, preprocessing using a configurable pipeline for analysis of connectomes to obtain BOLD sequences;
constructing a functional feature matrix using the BOLD sequences, and straightening an upper triangular portion of the functional feature matrix to obtain functional connectivity feature vectors, which are used as input to a deep multipath attention adaptive graph convolutional network (DMAGCN) model;
constructing the DMAGCN model, which consists of a backbone network and a plurality of branch networks; the backbone network is built using a Transformer, and the plurality of branch networks are constructed using Multi-Layer Perceptrons (MLPs) and graph networks, respectively; wherein the backbone network is used to extract common features from all source domains, and the plurality of branch networks are used to extract unique features from individual source domains;
validating the DMAGCN model using a five-fold cross-validation method to obtain an optimal DMAGCN model;
and inputting data to be classified into the optimal DMAGCN model to obtain data classification results.

Optionally, preprocessing involves skull stripping, slice timing correction, motion correction, global average intensity normalization, interference signal regression, bandpass filtering (0.01-0.1 Hz), and registration of resting-state functional magnetic resonance imaging data to standard anatomical positions.

Optionally, a formula for calculating the functional connectivity feature vectors is as follows:

$$corr ( {X}_{i , t} , {X}_{j , t} ) = \frac{{\sum }_{S = 1}^{T} ( {X}_{i , t} - {\overline{X}}_{i} ) ( {X}_{j , t} - {\overline{X}}_{j} )}{\sqrt{{\sum }_{S = 1}^{T} ( {X}_{i , t} - {\overline{X}}_{i} ) \sqrt{{\sum }_{S = 1}^{T} ( {X}_{j , t} - {\overline{X}}_{j} )}}} ;$$

Where Xᵢ and Xⱼ are average time series of i-th and j-th Region of Interest (ROI) regions; X_{i,t} and X_{j,t} are blood oxygen level-dependent (BOLD) intensities of Xᵢ and Xⱼ at time t; X̅ᵢ and X̅ⱼ represent means of average BOLD time series of i-th and j-th brain regions, respectively; T represents a total number of time points in an average BOLD sequence; and corr represents a Pearson correlation coefficient.

Optionally, the backbone network built from MLP and Transformer extracts features from the imaging data; in addition to imaging features, the graph network uses non-imaging data as edges between nodes to provide multimodal information for training the DMAGCN model and after training, the graph network is discarded, and other networks jointly make decisions about a target domain.

Optionally, the backbone network uses Transformer as the backbone network; an overall structure of Transformer comprises a multi-head self-attention module, a feedforward network, a residual connectivity layer, and a normalization layer.
a self-attention mechanism is the core of a Transformer-encoder and is calculated from a Query matrix, a Key matrix, and a Value matrix:

$$Attention ( Q , K , V ) = softmax ( \frac{Q {K}^{T}}{\sqrt{{D}_{k}}} ) V ;$$

Where Q∈ [symbol] ^{N×D}^{ₖ}, K∈ [symbol] ^{M×D}^{ₖ}, V∈ [symbol] ^{M×D}^{ᵥ}, N and M represent lengths of the Query matrix and the Key matrix, and Dₖ and Dᵥ represent dimensions of the Key matrix and Value matrix; Softmax is an activation function that converts attention scores into probabilities; the Transformer employs a multi-head attention mechanism:

$$MultiHead ( Q , K , V ) = Concat ( head 1 , … , {head}_{h} ) {W}^{0}$$

$${head}_{i} = Attention ( {QW}_{i}^{Q} , K {W}_{i}^{K} , {VW}_{i}^{V} )$$

Where

$${W}_{i}^{Q} ∈ {ℝ}^{N \times {D}_{k}} , {W}_{i}^{K} ∈ {ℝ}^{N \times {D}_{k}} , {W}_{i}^{V} ∈ {ℝ}^{M \times {D}_{v}}$$

are parameter matrices corresponding to Q,K,V; w° is a parameter matrix used for multi-head attention computation; then, the feedforward network applies two linear transformations with Gelu activation functions to an output of multi-head self-attention:

$$X = F F N ( x ) = G e l u ( x {W}_{1} + {b}_{1} ) {W}_{2} + {b}_{2} ;$$

Where x is an output of a previous layer, and W₁, W₂, b₁, and b₂ represent a training parameter matrix and bias values.

Optionally, to complete multi-task feature extraction, multiple branch networks are set up to extract unique features of each of the source domains; [symbol] , i=1, 2, . . . , N represents input features, which consist of fully connected layers, each with multiple nodes; each of the nodes receives the output of the previous layer node as an input; an output of a k-th node in an l-th layer is:

$${o}_{k}^{( l )} = {α}_{l} ( {\sum }_{j} {o}_{j}^{( l - 1 )} {θ}_{jk}^{( l )} ) ;$$

where

$${o}_{k}^{( l )}$$

is the output of the l-th layer; l=1, 2, . . . , L; k=1, 2, . . . , K; j=1, 2, . . . , J; k≠j;

$${o}_{j}^{( l - 1 )}$$

is a feature of a k-th node in a layer (l−1);

$${θ}_{jk}^{( l )}$$

represents a connection weight between k-th nodes in the l-th layer; αₗ represents an activation function of the l-th layer;
the common features of all the source domains are extracted using the backbone network:

$$= {f}_{c , i} ( {o}_{s} ; {Θ}_{c , i} ) ;$$

extracting the unique features of the i-th source domain using the plurality of branch networks:

$$= {f}_{s , i} ( {o}_{s} ; {Θ}_{s , i} ) ;$$

Where i=1, 2, 3 . . . I represents samples of the i-th source domain or the target domain; oₛ is a source domain dataset; f_{c,i}(⋅) is a common feature extractor; f_{s,i}(⋅) is a unique feature extractor; Θ_{s,i} are parameters of the i-th source domain unique feature extractor; Θ_{c,i} represent parameters of the i-th common feature extractor; [symbol] represent the common features of the samples of the i-th source domain in the source domains; [symbol] represent the unique features of the samples of the i-th source domain.

Optionally, maximum mean difference measure is used to measure a distance between distributions of source domain-related common features and unique features, specifically:

$${L}_{c o m} = MMD ( , ) = {‖ \frac{1}{n} {\sum }_{i = 1}^{n} - \frac{1}{n} {\sum }_{j = 1}^{n} ‖}_{H}^{2}$$

$${L}_{s} = MMD ( , ) = {‖ \frac{1}{n} {\sum }_{i = 1}^{n} - \frac{1}{n} {\sum }_{j = 1}^{n} ‖}_{H}^{2}$$

$${L}_{cs} = MMD ( , ) = {‖ \frac{1}{n} {\sum }_{i = 1}^{n} - \frac{1}{n} {\sum }_{j = 1}^{n} ‖}_{H}^{2}$$

Where n is a number of the samples in the source domains; i and j represent serial numbers of the samples, i≠j; and

$${‖ \cdot ‖}_{H}^{2}$$

represents Gaussian kernel Hilbert space.

A domain alignment loss is:

$${L}_{d o m a i n} = {L}_{c o m} + {L}_{s} + {L}_{c s} , ;$$

Optionally, the graph network is specifically an edge-variable graph convolutional network, utilizing spatial perception of the brain network and demographic relationships of a dataset to train and optimize a model.

Given data from N subjects consisting of the imaging data and the non-imaging data, a general graph is constructed: G=(V,E,W), where |v|=N represents a set of vertices, E⊆V×V is a set of edges, and weights of the edges are W; node features Zᵢ∈ [symbol] ^{C} are defined as C-dimensional feature vectors extracted from the imaging data of the i-th subject; the weights w_{i,j}∈W between (xᵢ, xⱼ) are defined as a learnable function representing information from the non-imaging data: φ: (xᵢ, xⱼ), which is modeled and trained by a pairwise association encoder PAE:

$${h}_{i} = ϕ ( {x}_{i} , Ω ) ;$$

$${h}_{j} = ϕ ( {x}_{j} , Ω ) ;$$

$${w}_{i , j} = \frac{{h}_{i}^{T} {h}_{j}}{2 ‖ {h}_{i} ‖ ‖ {h}_{j} ‖} + 0.5 ;$$

Where [symbol] is a normalized input; τ is a ReLU function; h; and h are mappings of the input features xᵢ and xⱼ in the same feature space; and Ω represents parameters trained in PAE;
an uncertainty-aware prediction loss function is:

$${L}_{e v} = - {\sum }_{i = 1}^{i = N} softmax ( P ( {x}_{i} ) , ) ;$$

Where P(xᵢ) represents a predicted value of the i-th sample, and [symbol] is a true value of the i-th sample; therefore, a total loss function of the DMAGCN model is:

$$λ = \frac{2}{1 + exp ( - γ \cdot ρ )} - 1 ;$$

$$L = λ {L}_{d o m a i n} + {L}_{e v} ;$$

Where λ varies from 0 to 1 over time, γ is a hyperparameter, ρ and represents a number of iterations.

Optionally, graph convolutional layers consist of Chebyshev convolutions, with a recurrence relation of the Chebyshev polynomial:

$${T}_{0} ( L ) = 1 , {T}_{1} ( L ) = L ;$$

$${T}_{k} ( L ) = 2 L {T}_{k - 1} ( L ) - {T}_{k - 2} ( L ) ;$$

a formula for Chebyshev convolution is:

$${H}^{l + 1} = {\sum }_{k = 0}^{K} {T}_{k} ( L ) {H}^{l} {θ}_{k}^{l} ;$$

Where, Tₖ(L) represents an expression of a topological structure L of a graph G after Chebyshev polynomial computation at term k; H^{l} represents a feature vector of a node at the layer l; and

$${θ}_{k}^{l}$$

represents convolution kernel parameters.

A data classification system based on a deep multipath attention adaptive graph convolutional network comprises:
a data acquisition and processing module, which acquires resting-state functional magnetic resonance imaging (fMRI) data from an autism brain imaging data exchange database, preprocesses using a configurable pipeline of connectom analysis, and obtains BOLD sequences;
a feature extraction module, which constructs a functional feature matrix using the BOLD sequences, and straightens an upper triangular portion of the functional feature matrix to obtain functional connectivity feature vectors, which are used as input to the DMAGCN model;
a model construction module, which constructs the DMAGCN model, which consists of a backbone network and multiple branch networks; wherein the backbone network is built using a Transformer, and the branch networks are constructed using MLP and graph networks, respectively; the backbone network is used to extract common features across all source domains, and the branch networks are used to extract features specific to individual source domains;
a model validation module, which validates the DMAGCN model using five-fold cross-validation to obtain an optimal DMAGCN model; and
a result output module, which inputs data to be classified into the optimal DMAGCN model to obtain data classification results.

As can be seen from the above technical solution, compared with the prior art, the present invention discloses a data classification method and system based on a deep multipath attention adaptive graph convolutional network. First, BOLD sequences are obtained from rs-fMRI data acquired and preprocessed from the ABIDE database. Functional connectivity feature vectors are then constructed and input into the DMAGCN model, which consists of a network with a specific structure. Finally, the optimal model is obtained through five-fold cross-validation for classification. The present invention ensures data quality through data preprocessing, laying the foundation for accurate analysis. Functional connectivity feature vectors effectively represent the data. After being input into the model, the Transformer backbone network and MLP branch networks can extract multi-source domain features. Combined with graph networks utilizing non-imaging data, the model can learn rich features, enhancing generalization and adaptability. Five-fold cross-validation ensures model reliability and improves classification accuracy, especially performing well in classifying uncertain data such as autism spectrum disorder, promoting research on related diseases and providing an efficient and reliable method for medical data classification.

### BRIEF DESCRIPTION OF THE DRAWINGS

To more clearly illustrate the technical solutions in the embodiments of the present invention or the prior art, the drawings used in the description of the embodiments or the prior art will be briefly introduced below. Obviously, the drawings described below are merely embodiments of the present invention. For those skilled in the art, other drawings can be obtained based on the provided drawings without creative effort.

FIG. 1 is a schematic diagram of a functional connection feature vector construction process provided by the present invention;

FIG. 2 is an overall structure diagram of a DMAGCN model provided by the present invention;

FIG. 3 is a backbone and branch network structure diagram provided by the present invention.

### DETAILED DESCRIPTION OF THE EMBODIMENTS

The technical solutions in the embodiments of the present invention will be clearly and completely described below with reference to the accompanying drawings. Obviously, the described embodiments are only some embodiments of the present invention, and not all embodiments. Based on the embodiments of the present invention, all other embodiments obtained by those skilled in the art without creative effort are within the protection scope of the present invention.

An embodiment of the present invention discloses a data classification method based on a deep multipath attention adaptive graph convolutional network, comprising: obtaining resting-state functional magnetic resonance imaging (fMRI) data from the Autism Brain Imaging Data Exchange (ABIDE) database,
preprocessing it using a configurable pipeline of connectom analysis to obtain BOLD sequences;
constructing a functional feature matrix using the BOLD sequences, and straightening the upper triangular portion of the functional feature matrix to obtain functional connectivity feature vectors, which are used as input to the DMAGCN model;
constructing the DMAGCN model, which consists of a backbone network and multiple branch networks; wherein the backbone network is built using a Transformer, and the branch networks are composed of MLPs and graph networks, respectively; the backbone network is used to extract common features of all source domains, and the branch networks are used to extract features specific to individual source domains;
validating the DMAGCN model using a five-fold cross-validation method to obtain the optimal DMAGCN model;
and inputting the data to be classified into the optimal DMAGCN model to obtain the data classification results.

In a specific embodiment, the dataset is provided by the Autism Brain Imaging Data Exchange (ABIDE) and has been preprocessed using a configurable pipeline of connectom analysis (C-PAC). The acquisition equipment was a Siemens system. The workflow involved skull stripping, slice timing correction, motion correction, global average intensity normalization, interference signal regression, bandpass filtering (0.01-0.1 Hz), and registration of fMRI images to standard anatomical space (MNI152). The dataset used came from six acquisition sites, including N=1735 subjects. Specific statistical information is shown in the table below:

TABLE 1
Dataset Information

| | | | | | |
|---|---|---|---|---|---|
| | No. of | | | | |
| | individ- | | Gender | TR/TE | Voxel |
| Site | uals | ASD/NC | (M/F) | (ms) | size (mm) |

AAL

| | | | | | |
|---|---|---|---|---|---|
| NYU | 184 | 79/105 | 147/37 | 2530/3.25 | 1.3 × 1.0 × 1.3 |
| USM | 101 | 58/43 | 101/0 | 2300/2.91 | 1.0 × 1.0 × 1.2 |
| UM | 145 | 68/77 | 117/28 | 5.7/250 | 3.4 × 3.4 × 3.0 |
| Leuven | 64 | 29/35 | 56/8 | 9.6/4.6 | 3.59 × 3.59 × 4.0 |
| YALE | 56 | 36/20 | 40/16 | 1230/1.73 | 1.0 × 1.0 × 1.0 |
| UCLA | 99 | 54/45 | 87/12 | 2300/2.84 | 1.0 × 1.0 × 1.2 |

CC200

| | | | | | |
|---|---|---|---|---|---|
| NYU | 174 | 74/100 | 138/36 | 2530/3.25 | 1.3 × 1.0 × 1.3 |
| USM | 67 | 43/24 | 67/0 | 2300/2.91 | 1.0 × 1.0 × 1.2 |
| UM | 120 | 47/73 | 93/27 | 5.7/250 | 3.4 × 3.4 × 3.0 |
| Leuven | 56 | 26/30 | 49/7 | 9.6/4.6 | 3.59 × 3.59 × 4.0 |
| YALE | 41 | 22/19 | 25/16 | 1230/1.73 | 1.0 × 1.0 × 1.0 |
| UCLA | 85 | 48/37 | 74/11 | 2300/2.84 | 1.0 × 1.0 × 1.2 |

Dosenbatch 160

| | | | | | |
|---|---|---|---|---|---|
| NYU | 174 | 74/100 | 138/36 | 2530/3.25 | 1.3 × 1.0 × 1.3 |
| USM | 67 | 43/24 | 67/0 | 2300/2.91 | 1.0 × 1.0 × 1.2 |
| UM | 120 | 47/73 | 93/27 | 5.7/250 | 3.4 × 3.4 × 3.0 |
| Leuven | 56 | 26/30 | 47/7 | 9.6/4.6 | 3.59 × 3.59 × 4.0 |
| YALE | 41 | 22/19 | 25/16 | 1230/1.73 | 1.0 × 1.0 × 1.0 |
| UCLA | 85 | 48/37 | 74/11 | 2300/2.84 | 1.0 × 1.0 × 1.2 |

Explanation of parameters in the table:
Repetition Time (TR): the time interval between consecutive pulse sequences applied to the same slice.
Echo Time (TE): the time interval between the excitation RF pulse and the peak of the detected echo signal.
Short TR and short TE enhance T1-weighted contrast, making tissues with short T1 relaxation times (e.g., fat) appear brighter, while tissues with longer T1 times (e.g., fluid) appear darker.
Longer TR and TE values produce T2-weighted images, where tissues with longer T2 relaxation times (e.g., fluid) appear brighter. Longer TR ensures full longitudinal relaxation, while longer TE provides sufficient lateral dephasing for T2 contrast.
Voxel Size: affects image resolution; smaller voxels result in higher resolution and also influence data analysis results. Excessively large voxels may fail to capture subtle differences between brain regions.

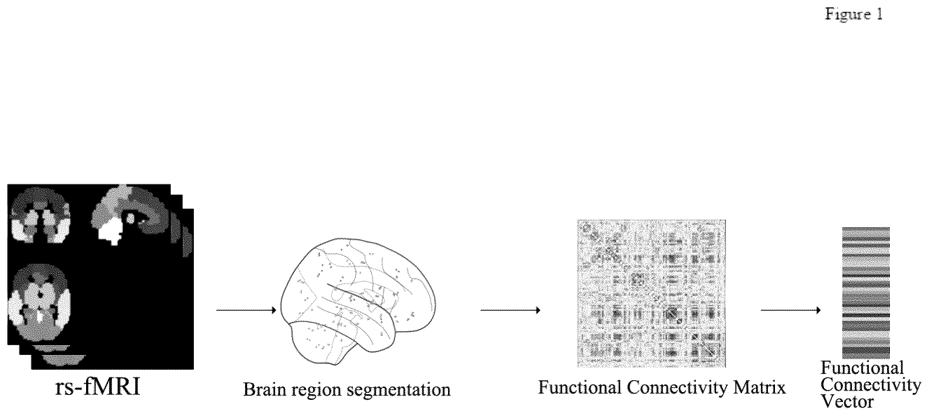

In a specific embodiment, the raw feature extraction is shown in FIG. 1. For each sample, the Pearson correlation coefficient between any pair of ROI regions in each brain is calculated to obtain the final functional connectivity feature matrix. Due to the symmetry of the matrix, only the upper triangular portion is selected, stretching it into a one-dimensional vector as the functional connectivity eigenvector. The formula for calculating the functional connectivity eigenvector is as follows:

$$corr ( {X}_{i , t} , {X}_{j , t} ) = \frac{{\sum }_{S = 1}^{T} ( {X}_{i , t} - {\overline{X}}_{i} ) ( {X}_{j , t} - {\overline{X}}_{j} )}{\sqrt{{\sum }_{S = 1}^{T} ( {X}_{i , t} - {\overline{X}}_{i} ) \sqrt{{\sum }_{S = 1}^{T} ( {X}_{j , t} - {\overline{X}}_{j} )}}} ;$$

Where Xᵢ and Xⱼ are the average time series of the i-th and j-th ROI regions; X_{i,t} and X_{j,t} are the BOLD intensities of Xᵢ and Xⱼ at time t; X̅ᵢ and X̅ⱼ represent the mean of the average BOLD time series of the i-th and j-th brain regions, respectively; T represents the total number of time points in the average BOLD sequence; and corr represents the Pearson correlation coefficient.

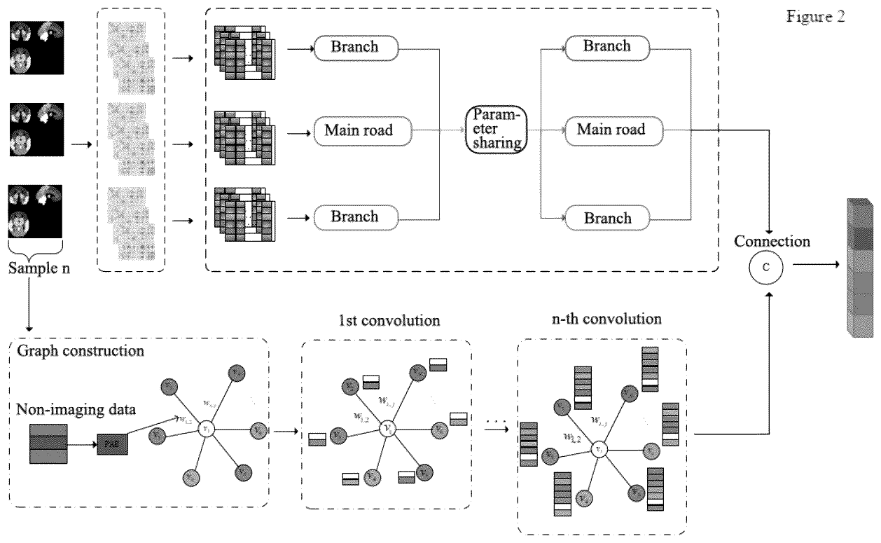

In a specific embodiment, the entire process flow is shown in FIG. 2, including four parts: data preprocessing, functional connectivity feature extraction, extraction of mixed features, and classification. Specifically, the rs-fMRI data is first preprocessed to obtain the BOLD sequence. Then, a functional feature matrix is constructed using the BOLD sequence, and the upper triangular part of the matrix is straightened to obtain the functional connectivity feature vector. Because the datasets involved come from different imaging centers, this is a multi-source domain problem. The model consists of a backbone network plus multiple branch networks. The backbone network is built using Transformer, and the branch networks are composed of MLP and graph networks, respectively. The network built using MLP and Transformer primarily extracts features from imaging data. In addition to imaging features, the graph network utilizes non-imaging information as edges between nodes to provide multimodal information for model training. The backbone network extracts common features from all source domains, while branch networks extract features specific to individual source domains. After training, the graph network is discarded, and the other networks collectively make decisions about the target domain.

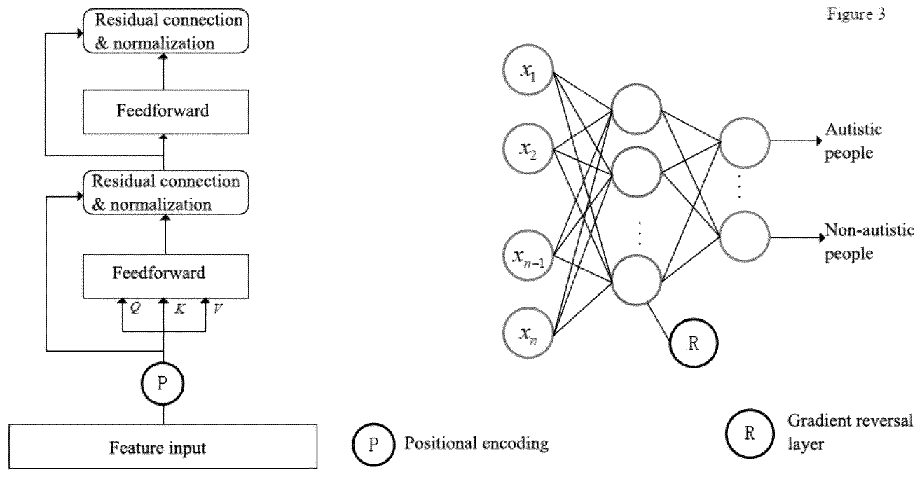

The branch network structure, as shown in FIG. 3, consists of fully connected layers. The input features are the output of the Transformer structure. Data undergoes dimensionality reduction in the deep neural network, from which the network learns feature information and extracts key features for subsequent model optimization and classification.

In a specific embodiment, the backbone network uses Transformer. The overall Transformer structure includes a multi-head self-attention module, a feedforward network, a residual connectivity layer, and a normalization layer.

The self-attention mechanism is the core of the Transformer encoder, calculated from the Query, Key, and Value matrices.

$$Attention ( Q , K , V ) = softmax ( \frac{Q {K}^{T}}{\sqrt{{D}_{k}}} ) V ;$$

Where Q∈ [symbol] ^{N×D}^{ₖ}, K∈ [symbol] ^{M×D}^{ₖ}, V∈ [symbol] ^{M×D}^{ᵥ}, N and M represent the lengths of the query and key, and Dₖ and Dᵥ represent the dimensions of the key and value; Softmax is an activation function that converts attention scores into probabilities; the Transformer employs a multi-head attention mechanism:

$$MultiHead ( Q , K , V ) = Concat ( head 1 , … , {head}_{h} ) {W}^{0}$$

$${head}_{i} = Attention ( {QW}_{i}^{Q} , K {W}_{i}^{K} , {VW}_{i}^{V} ) ;$$

Where

$${W}_{i}^{Q} ∈ {ℝ}^{N \times {D}_{k}} , {W}_{i}^{K} ∈ {ℝ}^{N \times {D}_{k}} , {W}_{i}^{V} ∈ {ℝ}^{M \times {D}_{v}}$$

are parameter matrices corresponding to Q,K,V; w° is the parameter matrix used for multi-head attention computation; then, the feedforward network applies two linear transformations with Gelu activation functions to the output of multi-head self-attention:

$$X = F F N ( x ) = G e l u ( x {W}_{1} + {b}_{1} ) {W}_{2} + {b}_{2} ;$$

Where x is the output of the previous layer, and W₁, W₂, b₁, and b₂ represent the training parameter matrix and bias values.

To accomplish multi-task feature extraction, multiple branch networks are set up to extract the unique features of each source domain. [symbol] , i=1, 2, . . . , N represents the input feature, which consists of fully connected layers. Each layer has multiple nodes, and each node receives the output of the previous layer node as input. The output of the k-th node in the l-th layer is:

$${o}_{k}^{( l )} = {α}_{l} ( {\sum }_{j} {o}_{j}^{( l - 1 )} {θ}_{jk}^{( l )} ) ;$$

Where

$${o}_{k}^{( l )}$$

is the output of layer l; l=1, 2, . . . , L; j=1, 2, . . . , J; k≠j;

$${o}_{j}^{( l - 1 )}$$

is the feature of the ^{k}th node in layer l−1;

$${θ}_{j k}^{( l )}$$

represents the connection weight between the k nodes in layer l; αₗ represents the activation function of the l-th layer; The common features of all source domains are extracted using the backbone network:

$$= {f}_{c , i} ( {o}_{s} ; {Θ}_{c , i} ) ;$$

Extract the unique features of the i-th source domain using a branch network:

$$= {f}_{s , i} ( {o}_{s} ; {Θ}_{s , i} ) ;$$

Where i=1, 2, 3 . . . I represents the i-th source domain sample or target domain sample; oₛ is the source domain dataset; f_{c,i}(⋅) is the common feature extractor; f_{s,i}(⋅) is the unique feature extractor; Θ_{s,i} are the parameters of the i-th source domain unique feature extractor; Θ_{c,i} represent the parameters of the i-th common feature extractor; [symbol] represent the common features of the i-th source domain sample in the source domain; and [symbol] represent the unique features of the i-th source domain sample.

Maximum mean discrepancy (MMD) is one of the most widely used loss functions in transfer learning, especially in domain adaptation. It is mainly used to measure the distance between the distributions of two different but related random variables. Here, MMD is used to measure the distance between the distributions of common and specific features related to the source domain. Specifically:

$${L}_{com} = MMD ( , ) = {‖ \frac{1}{n} {\sum }_{i = 1}^{n} - \frac{1}{n} {\sum }_{j = 1}^{n} ‖}_{H}^{2} ;$$

$${L}_{s} = MMD ( , ) = {‖ \frac{1}{n} {\sum }_{i = 1}^{n} - \frac{1}{n} {\sum }_{j = 1}^{n} ‖}_{H}^{2} ;$$

$${L}_{cs} = MMD ( , ) = {‖ \frac{1}{n} {\sum }_{i = 1}^{n} - \frac{1}{n} {\sum }_{j = 1}^{n} ‖}_{H}^{2} ;$$

Where n is the number of samples in the source domain; i and j represent the sample numbers, i≠j; and

$${‖ \cdot ‖}_{H}^{2}$$

represents the Gaussian kernel Hilbert space.

The domain alignment loss is:

$${L}_{domain} = {L}_{com} + {L}_{s} + {L}_{cs} .$$

In one specific embodiment, uncertainty-aware population graph construction, specifically using an edge-variable graph convolutional network, provides a method for constructing an adaptive population graph with partially labeled nodes and variational edges. This integrates imaging and non-imaging data from the population for uncertainty-aware disease prediction, and its effectiveness has been demonstrated. This model incorporates this as one branch, aiming to leverage the spatial perception of brain networks and population relationships within the dataset to train and optimize the model.

Given data from N subjects consisting of imaging and non-imaging data, a general graph is constructed: G=(V,E,W), where |V|=N represents the set of vertices, E⊆V×V is the set of edges, and the weights of the edges are W; node features Zᵢ∈ [symbol] ^{C} are defined as C-dimensional feature vectors extracted from the imaging data of the i-th subject; the weights w_{i,j}∈W between (xᵢ, xⱼ) are defined as a learnable function representing information from the non-imaging data: φ: (xᵢ, xⱼ) which is modeled and trained by the pairwise association encoder PAE:

$${h}_{i} = ϕ ( {x}_{i} , Ω ) ;$$

$${h}_{j} = ϕ ( {x}_{j} , Ω ) ;$$

$${w}_{i , j} = \frac{{h}_{i}^{T} {h}_{j}}{2 ‖ {h}_{i} ‖ ‖ {h}_{j} ‖} + 0.5 ;$$

Where [symbol] is the normalized input; τ is the ReLU function; hᵢ and hⱼ are the mappings of the input features and in the same feature space; Ω represents the parameters trained in PAE;

The convolutional layers of graph convolutions are composed of Chebyshev convolutions, and the recurrence relation of the Chebyshev polynomials is as follows:

$${T}_{0} ( L ) = 1 , {T}_{1} ( L ) = L ;$$

$${T}_{k} ( L ) = 2 {LT}_{k - 1} ( L ) - {T}_{k - 2} ( L ) ;$$

The formula for Chebyshev convolution is:

$${H}^{l + 1} = {\sum }_{k = 0}^{K} {T}_{k} ( L ) {H}^{l} {θ}_{k}^{l} ;$$

Where, Tₖ(L) represents the topological structure of graph G after Chebyshev polynomial computation at the term; H^{l} represents the node feature vector of the layer; and

$${θ}_{k}^{l}$$

are the convolution kernel parameters.
the uncertainty-aware prediction loss function is:

$${L}_{ev} = - {\sum }_{i = 1}^{i = N} soft max ( P ( {x}_{i} ) , ) ;$$

Where P(xᵢ) represents the predicted value of the i-th sample, and [symbol] is the true value of the i-th sample; therefore, the total loss function of the DMAGCN model is:

$$λ = \frac{2}{1 + exp ( - γ \cdot ρ )} - 1 ;$$

$$L = λ {L}_{domain} + {L}_{ev} ;$$

Where λ varies from 0 to 1 over time, γ is a hyperparameter, ρ and represents the number of iterations.

In a specific implementation example, the experimental results and analysis are also shown;

### Experimental Setup

The experimental data comes from the ABIDE database, using data from six sites: NYU, USM, UM, Leuven, YALE, and UCLA to construct a new dataset for the experiment. The experiment is based on five-fold cross-validation, a common validation scheme in many studies. In the experiment, one of the six imaging centers is sequentially selected as the target domain, and the rest are used as source domains. Each source domain is divided into five subsets (each subset has a similar number of samples, and the number of ASD patients and healthy controls in each subset is also approximately the same). In each fold of the cross-validation, four subsets from each independent source domain are selected as the labeled source domain training set, and the target domain data from all five subsets are selected as the unlabeled target domain training set. Target domain labels are not used during training, and only used during testing to evaluate the model's classification performance on the target domain. The experiment ensures that each comparison algorithm uses the same data partitioning as the proposed algorithm. The above process is repeated ten times, and the average value is used to evaluate each method. ACC, SEN, SPE, and AUC are used as evaluation metrics to quantitatively assess the classification performance of all methods.

### Performance Metrics Evaluation

Accuracy (ACC), sensitivity (SEN), specificity (SPE), and AUC are used to measure the classification performance of all relevant methods.

ACC represents the proportion of correctly classified samples to the total number of samples, SEN represents the proportion of correctly classified samples among samples that are truly ASD, and SPE represents the proportion of correctly classified samples among samples that are truly healthy controls. The higher the values of these three indicators, the better the classification performance of the model. The calculation methods for ACC, SEN, and SPE are shown in the following formulas:

$$ACC = \frac{TP + TN}{TP + TN + FP + FN} ;$$

$$SEN = \frac{TP}{TP + FN} ;$$

$$SPE = \frac{TN}{TN + FP} ;$$

In the above formula, TP is the number of true positives, i.e., the number of samples with a true label of ASD that were correctly predicted; FN is the number of false negatives, i.e., the number of samples with a true label of ASD that were incorrectly predicted; TN is the number of true negatives, i.e., the number of samples with a true label of healthy control that were correctly predicted; and FP is the number of false positives, i.e., the number of samples with a true label of healthy control that were incorrectly predicted.

### Experimental Summary

Comparison with State-of-the-Art Methods and Baselines

To fully verify the effectiveness of the method proposed in the present invention, its results were compared with the results of state-of-the-art methods.
ST-Transformer: proposes a linear spatiotemporal multi-head attention unit to obtain spatial and temporal representations of fMRI data. In addition, a Gaussian GAN-based data balancing method is introduced to address the data imbalance problem in real-world ASD datasets used for ASD subtype diagnosis.
ST-ASDNet: proposes two modules: Bidirectional Long Short-Term Memory Transformer (BLSTM-Transformer) and Fully Convolutional Network Transformer (FCN-Transformer), to obtain spatial and temporal features of fMRI data, respectively.
BrainNETTF: proposes an orthogonal clustering readout operation based on self-supervised soft clustering and orthogonal projection. This design considers underlying functional modules that determine similar behaviors between ROI groups, resulting in distinguishable cluster-aware node embeddings and information graph embeddings.
RGTNet: proposes a residual graph transformer network with FC learning, namely RGTNet. A graph encoder is designed to extract temporally correlated features with long-range dependencies, from which an interpretable FC matrix can be modeled. In addition, a residual technique is introduced to deepen the GCN architecture, thereby learning higher-level information.
AIMAFE: proposes a multi-atlas deep feature representation method based on stacked denoising autoencoders (SDA). A multilayer perceptron (MLP) and ensemble learning method are proposed to perform the final ASD identification task.
MDANN: captures the interrelationships in multimodal data (functional neuroimaging data and PC data) by integrating multilayer neural networks, attention mechanisms, and feature fusion.
PLSNet: designs a time series encoder for context-rich feature extraction, followed by a functional connectivity generator to model correlations with long-range dependencies. Position embedding technology is used to assign a unique identifier to each graph region. A sparse method is embedded to filter significant nodes during message propagation, which also helps reduce dimensionality complexity.
MVS-GCN: a graph structure learning algorithm that adaptively constructs clean brain networks through a supervised learning scheme. Compared to whole-brain networks, coarse graph representations are beneficial for brain network embedding learning and disease diagnosis. Furthermore, graph structure learning considers group-level consistency from subjects across multiple sites by highlighting indicative edges, thus eliminating noisy correlations in brain networks.
LRCDRl: using low-rank representation, it mitigates marginal distribution differences between domains by aligning the global structure of projected multi-site data. To reduce conditional distribution differences across all site data, LRCDR learns class-discriminative representations from data across multiple source and target domains to enhance within-class compactness and between-class separability of the projected data.

This implementation uses Support Vector Machines (SVM), Random Forests (RF), and Naïve Bayes classifiers (NB), which are commonly used as baselines in neuroimaging studies.
Support Vector Machine: a linear classifier that uses maximizing the margin in the feature space as its learning strategy. It introduces kernel methods to map the original features to a high-dimensional space, effectively making it a non-linear classifier. Here, the penalty parameter C=1, the kernel function is “linear”, and gamma=1.
Random Forest: using decision trees as basic units, the random forest integrates multiple decision trees through ensemble learning. Each decision tree is trained by randomly sampling data with replacement from the training set and randomly selecting a subset of features, ensuring the independence between different trees and improving the noise resistance of the random forest. Here, the number of decision trees in the random forest is n=100.
Naïve Bayes Classifier: based on Bayesian theory, it assumes that all features have conditionally independent Gaussian distributions. The Naïve Bayes algorithm learns the joint probability distribution of input and output data, and then uses Bayes' theorem to infer the label with the highest posterior probability as the prediction.

TABLE 2
Comparison of experimental results on ABIDE with various methods
ABIDE

| Method | ACC | SEN | SPE |
|---|---|---|---|
| Transformer (AAL) | 65.6 | 64.2 | 67.0 |
| ST-Transformer (AAL) | 67.9 | 65.6 | 70.2 |
| ST-ASDNET (AAL) | 65.2 | 59.38 | 70.7 |
| BrainNETTF (AAL) | 71 | 72.5 | 69.3 |
| Com-BrainTF (AAL) | 72.5 | 80.1 | 65.7 |
| RGTNet (AAL) | 73.4 | 70.8 | 71.9 |
| RGTNet (CC200) | 74.4 | 75.2 | 73.4 |
| AIMAFE (AAL) | 74.5 | 80.7 | 64.94 |
| MDANN (AAL) | 73.2 | 74.5 | 71.7 |
| PLSNet (AAL) | 72.4 | 71.6 | 71.3 |
| PLSNet (CC200) | 76.4 | 73.3 | 78.6 |
| MVS-GCN (AAL) | 68.9 | 69.1 | 63.15 |
| MVS-GCN (CC200) | 69.9 | 70.2 | 6305 |
| ASD-DiagNet (AAL) | 70.3 | 68.3 | 72.2 |
| LRCDR (AAL) | 73.1 | 71.0 | 75.1 |
| Ours + EV-graph (AAL) | 75.6 | 69.7 | 79.1 |
| OursW/O EV-graph (AAL) | 67.8 | 63.8 | 67.0 |
| Ours (cc200) | 73.6 | 64.9 | 79.6 |
| Ours (dosenbatch160) | 79.4 | 72.1 | 84.8 |

TABLE 3
Comparison with machine learning methods

| Baseline | ACC | SPE | SEN |
|---|---|---|---|
| SVM | 66.5 | 75.7 | 55.9 |
| NB | 63.5 | 76.9 | 48.0 |
| RF | 62.5 | 80.0 | 42.1 |
| ours | 75.6 | 69.7 | 79.1 |

Tables 2 and 3 show the average performance of 10 repetitions of 5-fold cross-validation. The proposed method outperforms all comparison methods in terms of accuracy, which was validated on three atlases: 75.6% on the AAL atlas, 73.6% on CC200, and 79.4% on Dosenbach160. Compared to Transformer-related models, the accuracy on AAL is 2.2% higher than that of the RGTNet model; compared to GCN-related models, improvements were observed on both AAL and CC200, reaching up to 6.7%; compared to domain adaptation methods, the accuracy improved by 2.5%. In summary, although other methods have demonstrated good performance in classification problems, they are not as effective as the proposed method in addressing the inconsistent feature space distribution of rs-fMRI data. The reasons for this are: 1. Insufficiently detailed feature extraction; the present invention divides features into common and unique features, considering a wider range of information. 2. The present invention uses a multi-network approach, setting up a separate network for each sample, reducing the problem of forgetting due to excessive knowledge in a single network. 3. The present invention constructs an uncertainty-aware population graph and integrates it into a multi-branch network. Compared to other methods, it considers non-imaging data and the relationships between samples, making the model more generalizable.

### Ablation Experiment

The present embodiment validates the effectiveness of each part of the model, and the results are shown in Table 4:

TABLE 4
Validation results of effectiveness

| Method | ACC | SPE | SEN | AUC |
|---|---|---|---|---|
| Without DANN | 71.1 | 78.9 | 60.9 | 75.2 |
| Without Lₛ | 73.3 | 77.9 | 63.8 | 78.1 |
| Without L_{cs} | 71.1 | 64.7 | 76.1 | 75.3 |
| Without Lₑᵥ | 67.8 | 73.0 | 63.8 | 75.8 |
| DANN + Lₛ + L_{cs} + Lₑᵥ | 75.6 | 69.7 | 79.1 | 75.9 |

Ablation experiments were conducted at six sites: UM, NYU, USM, Leuven, UCLA, and Yale. The results are analyzed as follows:
1. Compared to the overall model, removing the DANN component resulted in a 4.5% decrease in accuracy. DANN integrates the feature extractor and classifier, achieving domain alignment and classification in a simple way, and using an adversarial approach for domain alignment. Similarly, removing the branch network alignment resulted in a 2.3% decrease in accuracy, and removing the alignment between shared and specific features resulted in a 4.5% decrease, demonstrating effectiveness of feature refinement.
2. After removing the population graph, the accuracy decreased by 7.8%, the highest among all losses. This proves that using EV-GCN as a branch network, introducing the relationships between samples and non-imaging data, is very effective for ASD diagnosis and classification, enhancing the model's generalization ability.

The present invention proposes a novel multi-center domain adaptive neural network based on Transformer and population graphs. The Transformer helps model long-term dependencies between data in time-series data. The GCN network utilizes the population graph to combine imaging and non-imaging data to optimize the model and enhance its generalization ability. Furthermore, this paper sets up a backbone network and multiple branch networks during the feature learning process to extract shared and specific features of the source domain. This method attempts to introduce population graphs into multi-task ASD. The present invention is validated on datasets from six imaging centers (UM, NYU, USM, Leuven, Yale, and UCLA) in the ABIDE I dataset, achieving an accuracy of up to 79.4%. A data classification system based on a deep multi-path attention adaptive graph convolutional network includes:
A data acquisition and processing module, which obtains resting-state functional magnetic resonance imaging data from the Autism Brain Imaging Data Exchange database, and uses a configurable pipeline for connectome analysis for preprocessing to obtain BOLD sequences;
A feature extraction module, which uses the BOLD sequences to construct a functional feature matrix, and straightens the upper triangular part of the functional feature matrix to obtain a functional connectivity feature vector, which serves as the input to the DMAGCN model;
A model construction module, which constructs the DMAGCN model. The model consists of a backbone network and multiple branch networks. The backbone network is built using a Transformer, and the branch networks are composed of MLPs and graph networks, respectively. The backbone network is used to extract common features of all source domains, and the branch networks are used to extract unique features of individual source domains;
A model validation module, which validates the DMAGCN model based on a five-fold cross-validation method to obtain the optimal DMAGCN model; and
A result output module, which inputs the data to be classified into the optimal DMAGCN model to obtain the data classification results.

The various embodiments in this specification are described in a progressive manner, with each embodiment focusing on the differences from other embodiments. Similar parts between the embodiments can be referred to each other. For the apparatus disclosed in the embodiments, since it corresponds to the method disclosed in the embodiments, the description is relatively simple, and relevant details can be found in the method section.

The above description of the disclosed embodiments enables those skilled in the art to implement or use the present invention. Various modifications to these embodiments will be apparent to those skilled in the art, and the general principles defined herein may be implemented in other embodiments without departing from the spirit or scope of the invention. Therefore, the present invention will not be limited to the embodiments shown herein, but is to be accorded the broadest scope consistent with the principles and novel features disclosed herein.

1. A data classification method based on a deep multipath attention adaptive graph convolutional network, comprising:
acquiring resting-state functional magnetic resonance imaging (fMRI) data from an autism brain imaging data exchange database, preprocessing using a configurable pipeline for analysis of connectomes to obtain BOLD sequences;
constructing a functional feature matrix using the BOLD sequences, and straightening an upper triangular portion of the functional feature matrix to obtain functional connectivity feature vectors, which are used as input to a deep multipath attention adaptive graph convolutional network (DMAGCN) model;
constructing the DMAGCN model, which consists of a backbone network and a plurality of branch networks; the backbone network is built using a Transformer, and the plurality of branch networks are constructed using Multi-Layer Perceptrons (MLPs) and graph networks, respectively; wherein the backbone network is used to extract common features from all source domains, and the plurality of branch networks are used to extract unique features from individual source domains;
validating the DMAGCN model using a five-fold cross-validation method to obtain an optimal DMAGCN model;
and inputting data to be classified into the optimal DMAGCN model to obtain data classification results;
the backbone network built from MLP and Transformer extracts features from the imaging data; in addition to imaging features, the graph network uses non-imaging data as edges between nodes to provide multimodal information for training the DMAGCN model and after training, the graph network is discarded, and other networks jointly make decisions about a target domain.

2. The data classification method based on a deep multipath attention adaptive graph convolutional network according to claim 1, wherein preprocessing involves skull stripping, slice timing correction, motion correction, global average intensity normalization, interference signal regression, bandpass filtering (0.01-0.1 Hz), and registration of resting-state functional magnetic resonance imaging data to standard anatomical positions.

3. The data classification method based on a deep multipath attention adaptive graph convolutional network according to claim 1, wherein a formula for calculating the functional connectivity feature vectors is as follows:
$$corr ( {X}_{i , t} , {X}_{j , t} ) = \frac{{\sum }_{S = 1}^{T} ( {X}_{i , t} - {\overline{X}}_{i} ) ( {X}_{j , t} - {\overline{X}}_{j} )}{\sqrt{{\sum }_{S = 1}^{T} ( {X}_{i , t} - {\overline{X}}_{i} ) \sqrt{{\sum }_{S = 1}^{T} ( {X}_{j , t} - {\overline{X}}_{j} )}}} ;$$
where Xᵢ and Xⱼ are average time series of i-th and j-th Region of Interest (ROI) regions; X_{i,t} and X_{j,t} are blood oxygen level-dependent (BOLD) intensities of Xᵢ and Xⱼ at time t; X̅ᵢ and X̅ⱼ represent means of average BOLD time series of i-th and j-th brain regions, respectively; T represents a total number of time points in an average BOLD sequence; and corr represents a Pearson correlation coefficient.

4. The data classification method based on a deep multipath attention adaptive graph convolutional network according to claim 1, wherein the backbone network uses Transformer as the backbone network; an overall structure of Transformer comprises a multi-head self-attention module, a feedforward network, a residual connectivity layer, and a normalization layer;
a self-attention mechanism is the core of a Transformer-encoder and is calculated from a Query matrix, a Key matrix, and a Value matrix:
$$Attention ( Q , K , V ) = soft max ( \frac{{QK}^{T}}{\sqrt{{D}_{k}}} ) V ;$$
where Q∈ [symbol] ^{N×D}^{ₖ}, K∈ [symbol] ^{M×D}^{ₖ}, V∈ [symbol] ^{M×D}^{ᵥ}, N and M represent lengths of the Query matrix and the Key matrix, and Dₖ and Dᵥ represent dimensions of the Key matrix and Value matrix; Softmax is an activation function that converts attention scores into probabilities; the Transformer employs a multi-head attention mechanism:
$$MultiHead ( Q , K , V ) = Concat ( head 1 , … , {head}_{h} ) {W}^{0}$$
$${head}_{i} = Attention ( {QW}_{i}^{Q} , {KW}_{i}^{K} , {VW}_{i}^{V} ) ;$$
where
$${W}_{i}^{Q} ∈ {ℝ}^{N \times {D}_{k}} , {W}_{i}^{K} ∈ {ℝ}^{N \times {D}_{k}} , {W}_{i}^{V} ∈ {ℝ}^{M \times {D}_{v}}$$
are parameter matrices corresponding to Q,K,V; w° is a parameter matrix used for multi-head attention computation; then, the feedforward network applies two linear transformations with Gelu activation functions to an output of multi-head self-attention:
$$X = FFN ( x ) = Gelu ( {xW}_{1} + {b}_{1} ) {W}_{2} + {b}_{2} ;$$
where x is an output of a previous layer, and W₁, W₂, b₁, and b₂ represent a training parameter matrix and bias values.

5. The data classification method based on a deep multipath attention adaptive graph convolutional network according to claim 1, wherein to complete multi-task feature extraction, multiple branch networks are set up to extract unique features of each of the source domains; [symbol] , i=1, 2, . . . , N represents input features, which consist of fully connected layers, each with multiple nodes; each of the nodes receives the output of the previous layer node as an input; an output of a k-th node in an l-th layer is:
$${o}_{k}^{( l )} = {α}_{l} ( {\sum }_{j} {o}_{j}^{( l - 1 )} {θ}_{jk}^{( l )} ) ;$$
where
$${o}_{k}^{( l )}$$
is the output of the l-th layer; l=1, 2, . . . , L; k=1, 2, . . . , K; j=1, 2, . . . , J; k≠j;
$${o}_{j}^{( l - 1 )}$$
is a feature of a k-th node in a layer (l−1);
$${θ}_{j k}^{( l )}$$
represents a connection weight between k-th nodes in the l-th layer; αₗ represents an activation function of the l-th layer;
the common features of all the source domains are extracted using the backbone network:
$$= {f}_{c , i} ( {o}_{s} , ; {Θ}_{c , i} ) ;$$
extracting the unique features of the i-th source domain using the plurality of branch networks:
$$0 = {f}_{s , i} ( {o}_{s} , ; {Θ}_{s , i} ) ;$$
where i=1, 2, 3 . . . I represents samples of the i-th source domain or the target domain; oₛ is a source domain dataset; f_{c,i}(⋅) is a common feature extractor; f_{s,i}(⋅) is a unique feature extractor; Θ_{s,i} are parameters of the i-th source domain unique feature extractor; Θ_{c,i} represent parameters of the i-th common feature extractor; [symbol] represent the common features of the samples of the i-th source domain in the source domains; [symbol] represent the unique features of the samples of the i-th source domain.

6. The data classification method based on a deep multipath attention adaptive graph convolutional network according to claim 5, wherein maximum mean difference measure is used to measure a distance between distributions of source domain-related common features and unique features, specifically:
$${L}_{c o m} = MMD ( ( , ) = {‖ \frac{1}{n} {\sum }_{j = 1}^{n} - \frac{1}{n} {\sum }_{j = 1}^{n} ‖}_{H}^{2} ;$$
$${L}_{s} = MMD ( ( , ) = {‖ \frac{1}{n} {\sum }_{j = 1}^{n} ( - \frac{1}{n} {\sum }_{j = 1}^{n} ‖}_{H}^{2} ;$$
$${L}_{c s} = MMD ( ( , ) = {‖ \frac{1}{n} {\sum }_{i = 1}^{n} - \frac{1}{n} {\sum }_{{j = 1}^{}}^{n} ‖}_{H}^{2} ;$$
where n is a number of the samples in the source domains; i and j represent serial numbers of the samples, i≠j; and
$${‖ \cdot ‖}_{H}^{2}$$
represents Gaussian kernel Hilbert space;
and a domain alignment loss is:
$${L}_{domain} = {L}_{c o m} + {L}_{s} + {L}_{c s} ;$$

7. The data classification method based on a deep multipath attention adaptive graph convolutional network according to claim 6, wherein the graph network is specifically an edge-variable graph convolutional network, utilizing spatial perception of the brain network and demographic relationships of a dataset to train and optimize a model;
given data from N subjects consisting of the imaging data and the non-imaging data, a general graph is constructed: G=(V,E,W), where |V|=N represents a set of vertices, E⊆V×V is a set of edges, and weights of the edges are W; node features Zᵢ∈ [symbol] ^{C} are defined as C-dimensional feature vectors extracted from the imaging data of the i-th subject; the weights w_{i,j}∈W between (xᵢ, xⱼ) are defined as a learnable function representing information from the non-imaging data: φ: (xᵢ, xⱼ), which is modeled and trained by a pairwise association encoder PAE:
$${h}_{i} = ϕ ( {x}_{i} , Ω ) ;$$
$${h}_{j} = ϕ ( {x}_{j} , Ω ) ;$$
$${w}_{i , j} = \frac{{h}_{i}^{T} {h}_{j}}{2 ‖ {h}_{i} ‖ ‖ {h}_{j} ‖} + 0.5 ;$$
where [symbol] is a normalized input; τ is a ReLU function; hᵢ and hⱼ are mappings of the input features xᵢ and xⱼ in the same feature space; and Ω represents parameters trained in PAE;
an uncertainty-aware prediction loss function is:
$${L}_{e v} = - {\sum }_{i = 1}^{i = N} soft max ( P ( {x}_{i} ) , ) ;$$
where P(xᵢ) represents a predicted value of the i-th sample, and [symbol] is a true value of the i-th sample;
therefore, a total loss function of the DMAGCN model is:
$$λ = \frac{2}{1 + exp ( - γ \cdot ρ )} - 1 ;$$
$$L = λ {L}_{d o m a i n} + {L}_{e v} ;$$
where λ varies from 0 to 1 over time, γ is a hyperparameter, ρ and represents a number of iterations.

8. The data classification method based on a deep multipath attention adaptive graph convolutional network according to claim 7, wherein graph convolutional layers consist of Chebyshev convolutions, with a recurrence relation of the Chebyshev polynomial:
$${T}_{0} ( L ) = 1 , {T}_{1} ( L ) = L ;$$
$${T}_{k} ( L ) = 2 {LT}_{k - 1} ( L ) - {T}_{k - 2} ( L ) ;$$
a formula for Chebyshev convolution is:
$${H}^{l + 1} = {\sum }_{k = 0}^{K} {T}_{k} ( L ) {H}^{l} {θ}_{k}^{l} ;$$
wherein, Tₖ(L) represents an expression of a topological structure L of a graph G after Chebyshev polynomial computation at term k; H^{l} represents a feature vector of a node at the layer l; and
$${θ}_{k}^{l}$$
represents convolution kernel parameters.

9. A data classification system based on a deep multipath attention adaptive graph convolutional network, by using the data classification method based on a deep multipath attention adaptive graph convolutional network according to claim 1, wherein comprises:
a data acquisition and processing module, which acquires resting-state functional magnetic resonance imaging (fMRI) data from an autism brain imaging data exchange database, preprocesses using a configurable pipeline of connectom analysis, and obtains BOLD sequences;
a feature extraction module, which constructs a functional feature matrix using the BOLD sequences, and straightens an upper triangular portion of the functional feature matrix to obtain functional connectivity feature vectors, which are used as input to the DMAGCN model;
a model construction module, which constructs the DMAGCN model, which consists of a backbone network and multiple branch networks; wherein the backbone network is built using a Transformer, and the branch networks are constructed using MLP and graph networks, respectively; the backbone network is used to extract common features across all source domains, and the branch networks are used to extract features specific to individual source domains;
a model validation module, which validates the DMAGCN model using five-fold cross-validation to obtain an optimal DMAGCN model; and
a result output module, which inputs data to be classified into the optimal DMAGCN model to obtain data classification results.
